(19) United States
(12) Plant Patent Application Publication
(10) Pub. No.: US 2013/0167275 P1
(43) Pub. Date: Jun. 27, 2013

(54) Variety of Calla Lily Named 'PACO'

(50) ;

(71) Applicant: Cecilius Jan-Jochem Randag, Bosweg 10 (NL)

(72) Inventor: Cecilius Jan-Jochem Randag, Bosweg 10 (NL)

(73) Assignee: SANDE BV, Bosweg 46B (NL)

(21) Appl. No.: 13/374,311

(22) Filed: Dec. 21, 2011

(51) Int. Cl. A01H 5/00

(52) U.S. Cl. PLT/313

(57) ABSTRACT

‘Paco’ is a new variety of calla lily having flowers with a purple-colored spathe that produces 2-15 flowers per tuber. The flower stems are about 50 cm. in height and the leaves are green with a few whitish spots and have a leathery texture.

### BOTANICAL CLASSIFICATION

Zantedeschia sprengeri

### VARIETAL DENOMINATION

‘PACO’

### BACKGROUND OF THE NEW PLANT

The present invention comprises a new and distinct cultivar of calla lily, botanically known as Zantedeschia sprengeri and hereinafter referred to by the cultivar name ‘Paco’.

‘Paco’ is a product of a planned breeding program which had the objective of creating Zantedeschia hybrids for pot flower production in a wide range of colors with a large, classic flower shape. The breeding program began in 1989 and the new cultivar is a seedling selected from the crossing of Zantedeschia sprengeri seedling C02029 (male parent, unpatented) with an unnamed Zantedeschia sprengeri rehmanii selection (female parent, unpatented). ‘Paco’ was selected in 2004 by the inventor C. J. J. Randag in 't Zand, The Netherlands as one flowering plant within the progeny of the stated cross.

The first act of asexual reproduction of ‘Paco’ by tissue culture was performed by the inventor in August of 2005 in 't Zand, The Netherlands. Further asexual reproductions by tissue culture at the same location has demonstrated that the combination of characteristics as herein disclosed for the new cultivar are retained and reproduced true to type through successive generations of asexual reproduction.

The following observations, measurements and comparisons describe plants grown in 14 cm. pots in 't Zand, The Netherlands under greenhouse conditions which approximate those generally used in horticultural practice. Color references are made to the R.H.S. Colour Chart of The Royal Horticulture Society of London, except where general color terms of ordinary significance are used.

The present invention has not been evaluated under all possible environmental conditions. The phenotype may vary with variation in environment without a change in the genotype of the plant.

The following traits have been repeatedly observed and determined to basic characteristics of ‘Paco’ which, in combination, distinguish this calla lily as a new and distinct cultivar:

1. Purple-colored spathe;

2. Fast forcing time; and

3. High flower production.

TABLE 1
provides similarities and differences of ‘Paco’ to its parents and ‘Samur’.

| ‘Paco’ | Similarities | Differences |
|---|---|---|
| Z. rehmanii selection | Lear shape | Color of spathe |
| (female parent) | | |
| C02029 | Leaf shape | Number of spathes |
| (male parent) | | Color of spathe |
| ‘Samur’ | Leaf shape | Color of spathe |
| | | Spathe shape |

### BRIEF DESCRIPTION OF THE DRAWINGS

The accompanying photographic drawings illustrate the new variety, with the colors being as nearly true as is possible with color illustrations of this type:

FIG. 1 is a photograph of an entire plant of the new variety;

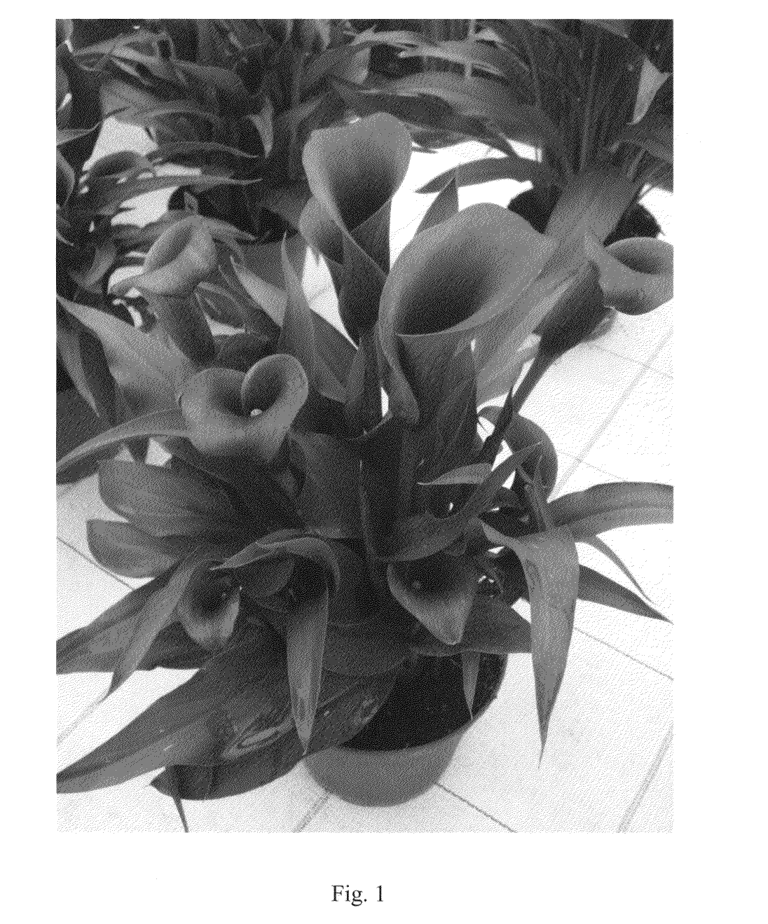

FIG. 2 is a close-up photograph of multiple plants of the new variety; and

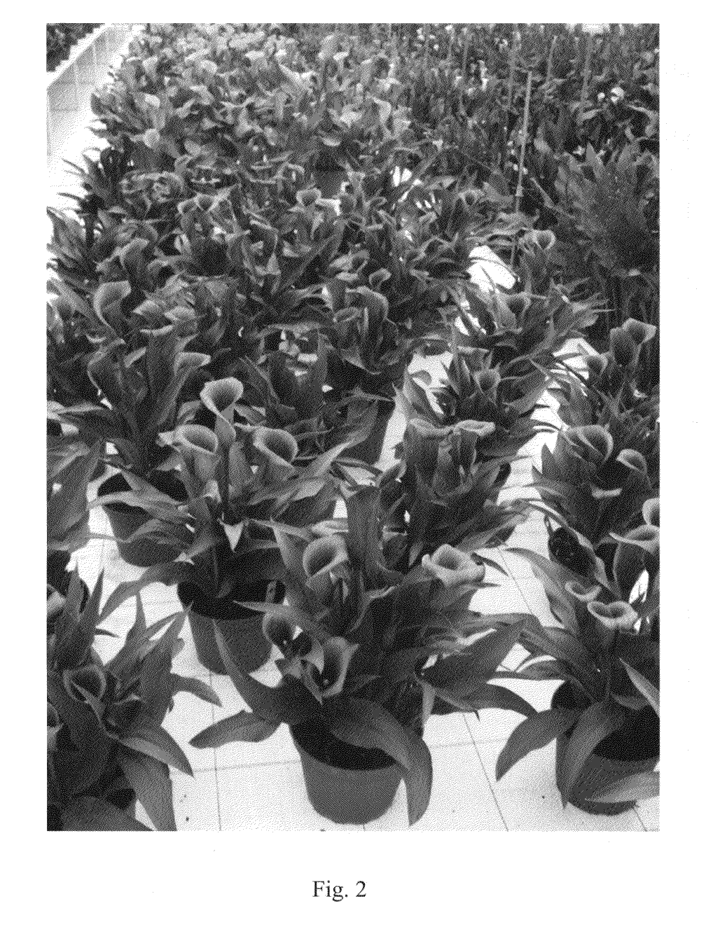

FIG. 3 is a photograph of an overhead view of the new variety.

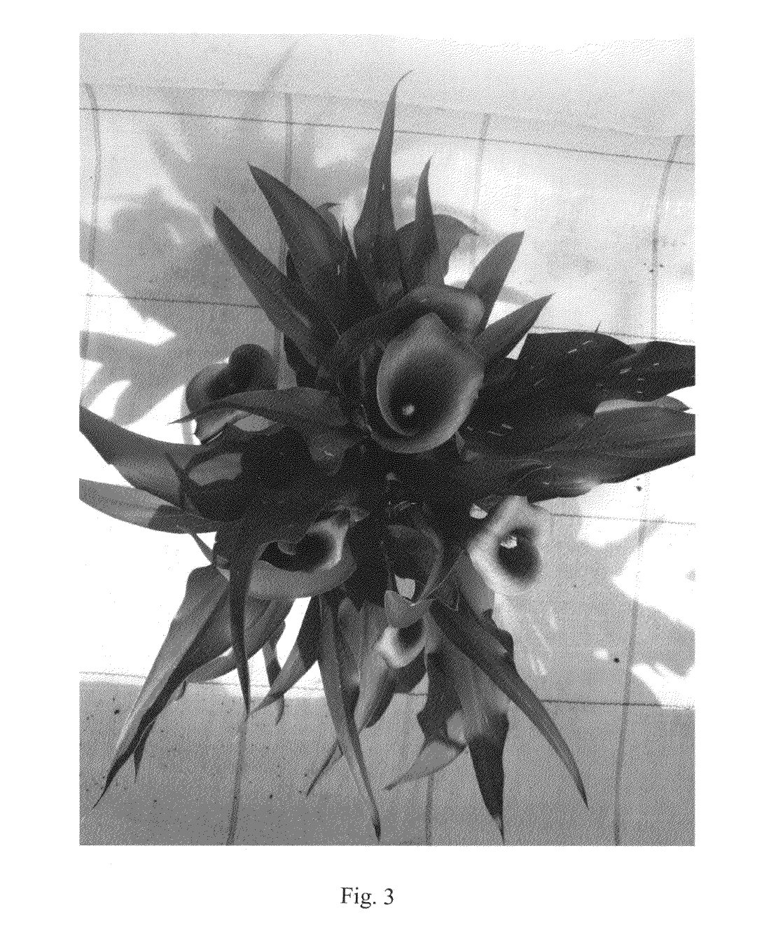

### DESCRIPTION OF THE PLANT

The plant:
   Size.—Height of the leaf canopy above the soil: 20-30 cm. Height of top of flower above the soil: 15-30 cm. Diameter: 10-20 cm.
   Form.—Erect.
   Number of flowers per tuber.—Size 9-12 cm. in diameter: 2-5 flowers. Size 12-15 cm. in diameter: 5-7 flowers. Size 15-18 cm. in diameter: 7-15 flowers.
   Branches.—Character: Many, like the variety ‘Chrystal Blush’ (unpatented). Color: Between Yellow-Green Group RHS 146A and 146D.
   Leaves.—Size: Width: 3-6 cm. Length: 25-40 cm. Number per plant: 20-30. Shape: Elliptic. Color: Upper surface: Green Group RHS 137A to 137B. Lower surface: Green Group RHS 136A to 137B. Margin: Wavy. Veins: Configuration: Pinnate. Color: Yellow-Green Group RHS 146A. Surface quality: Leathery. Petiole: Length: 10-30 cm. Color: The base is Green Group 138A and the upper part is between Yellow-Green Group RHS 144A and 144B.
   Roots.—Color: White. Branching: Similar to other Zantedeschia varieties.
The flower and seeds:
   Spathe.—Size: Length: 4-8 cm. Width: 4-6 cm. Color: Upper surface: Varies between Purple Group RHS N79B and N79D. Lower surface: Varies between Purple Group RHS N79B and N79D. Vein color: Purple Group RHS N29B. Shape: Cupped.
   Spadix.—Size: Length: 10-30 mm. Diameter: 2-5 mm. Color: Between Yellow-Orange Group RHS 16B and 16C. Position relative to spathe: Upright.
   Peduncle.—Size: Length: 30-65 cm. Diameter: 6-10 mm. Color: Upper part varies between Yellow-Green Group RHS 144A and 144B; the lower part is Green-White Group RHS 157D.
   Reproductive organs.—Location of female organs: Basal position of the spadix. Location of male organs: Upper position of the spadix. Perianth: Conspicuous. Stamens: Presence: Not visible before pollen release. Number: More than 20. Pollen amount: Similar to ‘Picasso’ (U.S. Plant Pat. No. 15,282). Color: Between White Group RHS 155A and 155B. Anther: Shape: Round and wire-like. Length: Less than 1 mm. Color: Between White Group RHS 155A and 155B. Filament: Length: Less than 1 mm. Color: Between Yellow-Orange Group RHS 16B and 16C. Pistil: Number: 20 to 40. Length beyond perianth: About 1 mm. Stigma: Shape: Round. Size: Less than 1 mm. Style: Length: Less than 1 mm. Color: Between Purple Group RHS N79B and N79D. Ovaries: Texture: Smooth. Length: Between 1 to 10 mm. Width: Between 1 to 10 mm. Color: Top color is between Purple Group RHS N79B and N79D; bottom color is between White Group RHS 155B and Green-White Group 157C. Seeds: Length: 4-7 mm. Width: 3-6 mm. Color: Between Purple Group RHS N79B and N79D. Amount: Between 1-50.
General:
   Disease resistance.—No unusual susceptibility to diseases noticed to date.
   Pest resistance.—No unusual susceptibility to pests noticed to date.
   Heat tolerance.—No heat tolerance.
   Hardiness.—Not hardy.
   Flowering.—Tubers planted in a greenhouse in The Netherlands in February produce flowers in April. Tubers planted outdoors in The Netherlands in May produce flowers in July.
   Lastingness.—About two weeks on the plant.
   Fragrance.—None.

I claim:

1. A new and distinct variety of calla lily plant named ‘Paco’ as herein described and illustrated.
